(19) United States
(12) Patent Application Publication
(10) Pub. No.: US 2022/0104803 A1
(43) Pub. Date: Apr. 7, 2022

(54) SUTURE PASSER INCLUDING AN ELECTROMAGNET

(71) Applicant: Covidien LP, Mansfield, MA (US)

(72) Inventors: Kevin M. Desjardin, Prospect, CT (US); Astley Collin Lobo, West Haven, CT (US)

(21) Appl. No.: 17/064,917

(22) Filed: Oct. 7, 2020

(51) Int. Cl. A61B 17/04; A61B 17/29

(52) U.S. Cl. CPC A61B 17/0482; A61B 17/0469; A61B 2017/00367; A61B 2017/047; A61B 17/2909

(57) ABSTRACT

A surgical kit includes a suture passer, a suture, a surgical access device, and a guide. The suture passer includes a handle assembly, an elongate shaft, a jaw assembly, and a suture retrieval assembly. The suture retrieval assembly includes an electromagnet disposed in the jaw assembly, a power module electrically coupled to the electromagnet, and an activation switch disposed on the handle assembly and electrically coupled to the power module to selectively activate the electromagnet. The suture includes a metal portion. A surgical access device includes a housing and a cannula extending distally from the housing. The cannula defines a lumen therethrough and lateral slots in communication with the lumen. The guide is detachably positionable with the surgical access device. The guide includes first and second channels and defines lateral openings in communication with the respective first and second channels. The lateral openings are in registration with the lateral slots.

### BACKGROUND

### Technical Field

The disclosure relates to surgical instruments and, more particularly, to a suture passer that electromagnetically retrieves a suture within a body cavity.

### Background of Related Art

In minimally invasive surgical procedures, including endoscopic and laparoscopic surgeries, a surgical access device permits the introduction of a variety of surgical instruments into a body cavity or opening. A surgical access device (e.g., a cannula) is introduced through an opening in tissue (i.e., a naturally occurring orifice or an incision) to provide access to an underlying surgical site in the body. The incision is typically made using an obturator having a blunt or sharp tip that has been inserted within the passageway of the surgical access device. For example, a cannula has a tube of rigid material with a thin wall construction, through which an obturator may be passed. The obturator is utilized to penetrate a body wall, such as an abdominal wall, or to introduce the surgical access device through the body wall, and is then removed to permit introduction of surgical instrumentation through the surgical access device to perform the surgical procedure.

Closing the incision, which may be referred to as a fascial closure, is generally accomplished by using a suture and is performed using separate instruments. Accordingly, there is a need for a suture passer that can retrieve a suture within a body cavity without relying on other surgical instruments such as, e.g., a surgical grasper, to close the incision, thereby reducing the number of openings in the patient during the minimally invasive surgical procedure.

### SUMMARY

The present disclosure describes a suture passer that demonstrates a practical approach to meeting the performance requirements and overcoming usability challenges associated with fascial closure procedures.

In accordance with the present disclosure, a surgical kit includes a suture passer, a suture, a surgical access device, and a guide The suture passer includes a handle assembly, an elongate shaft extending from the handle assembly and defining a lumen therethrough, a jaw assembly including first and second jaws transitionable between open and closed configurations, and a suture retrieval assembly. The suture retrieval assembly includes an electromagnet disposed in the jaw assembly, a power module electrically coupled to the electromagnet, and an activation switch disposed on the handle assembly that is electrically coupled to the power module to selectively activate the electromagnet. The suture includes a metal portion. The surgical access device includes a housing and a cannula extending distally from the housing. The cannula defines a lumen therethrough and defines lateral slots in communication with the lumen. The lateral slots diametrically oppose each other. The guide is detachably positionable within the surgical access device. The guide includes first and second channels and defines lateral openings in communication with the respective first and second channels. The lateral openings are in registration with the respective lateral slots of the surgical access device.

In aspects, the first jaw may be stationary relative to the elongate shaft.

In aspects, the first jaw may include a sharp tip configured to penetrate tissue.

In aspects, the first jaw may define a recess. The electromagnet may be disposed in the recess.

In aspects, the electromagnet may include a planar surface.

In aspects, the second jaw may be pivotably supported on the first jaw.

In aspects, the first or second channels of the guide may define an acute angle with respect to a longitudinal axis defined by the guide.

In aspects, the handle assembly may include a fluid port in communication with the jaw assembly.

In aspects, the power module may include a battery and a printed circuit board including a processor.

In accordance with another aspect of the disclosure, a suture passer for fascial closure includes a handle assembly including a pair of handles, an elongate shaft extending from the handle assembly and defining a lumen therethrough, a jaw assembly including first and second jaws transitionable between open and closed configurations, an actuation rod, a suture, and a suture retrieval assembly. The actuation rod interconnects the pair of handles and the jaw assembly such that actuation of the pair of handles transitions the jaw assembly between the open and closed configurations. The suture includes a metal portion. The suture retrieval assembly includes an electromagnet associated with the jaw assembly, a power module in electrical communication with the electromagnet, and an activation switch to selectively activate electromagnet to attract the metal portion of the suture thereto.

In aspects, the electromagnet may be disposed on one of the first or second jaws.

In aspects, the pair of handles may include a stationary handle and a movable handle pivotably supported on the stationary handle. The power module of the suture retrieval assembly may be disposed on the stationary handle.

In aspects, the first jaw may be fixedly secured to the elongate shaft and the second jaw may be pivotably supported on the first jaw.

In aspects, the first jaw may have a pointed tip to penetrate tissue.

In aspects, the metal portion of the suture may be formed of nickel or cobalt.

In accordance with yet another aspect of the disclosure, a suture passer includes a handle assembly including first and second handles, an elongate shaft extending from the handle assembly and defining a lumen therethrough, a jaw assembly including first and second jaws transitionable between open and closed configurations, and a suture retrieval assembly including an electromagnet disposed on the first jaw, a battery disposed on the first handle and in electrical communication with the electromagnet, and an activation switch to selectively activate the electromagnet.

### BRIEF DESCRIPTION OF THE DRAWINGS

Various aspects of the present disclosure are described hereinbelow with reference to the drawings, which are incorporated and constitute a part of this specification, wherein:

FIG. 1 is a perspective view of a suture passer in accordance with the present disclosure;

FIG. 2 is a side cross-sectional view of the suture passer of FIG. 1 taken along section line 2-2 of FIG. 1;

FIG. 3 is an enlarged perspective view of the indicated area of detail of FIG. 1;

FIG. 4 is an enlarged perspective view of the indicated area of detail of FIG. 1;

FIG. 5 is a partial perspective view of a suture for use with the suture passer of FIG. 1;

FIG. 6 is a perspective view of a fascial closure system utilizing the suture passer of FIG. 1;

FIGS. 7-10 are perspective views of the fascial closure system of FIG. 6 illustrating use thereof;

FIG. 11 is a cross-sectional view of tissue after performing the fascial closure utilizing the fascial closure system of FIG. 6.

### DETAILED DESCRIPTION

The disclosed suture passer is described in detail with reference to the drawings, in which like reference numerals designate identical or corresponding elements in each of the several views. As used herein, the term “distal,” as is conventional, will refer to that portion of the instrument, apparatus, device, or component thereof which is farther from the user, while the term “proximal” will refer to that portion of the instrument, apparatus, device, or component thereof which is closer to the user. As used herein, the terms parallel and perpendicular are understood to include relative configurations that are substantially parallel and substantially perpendicular up to about + or −10 degrees from true parallel and true perpendicular. In the following description, well-known functions or constructions are not described in detail to avoid obscuring the disclosure in unnecessary detail.

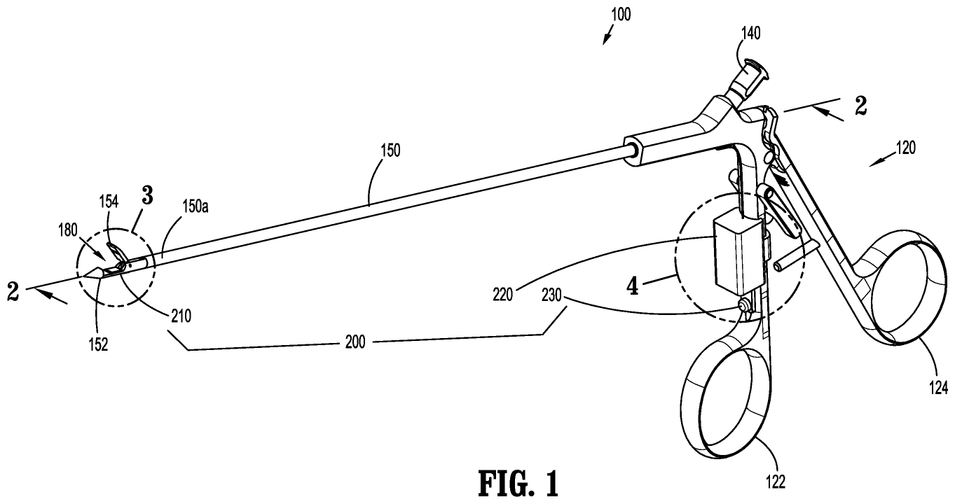

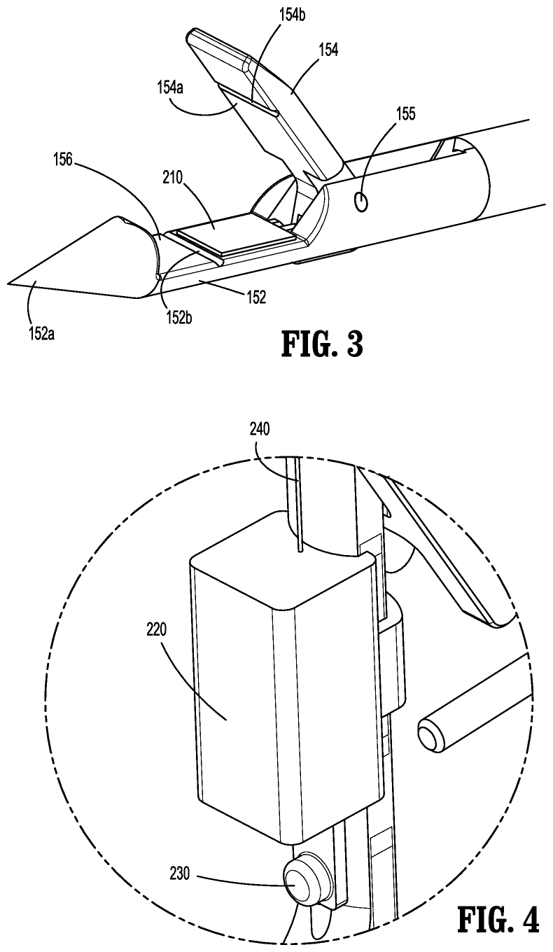

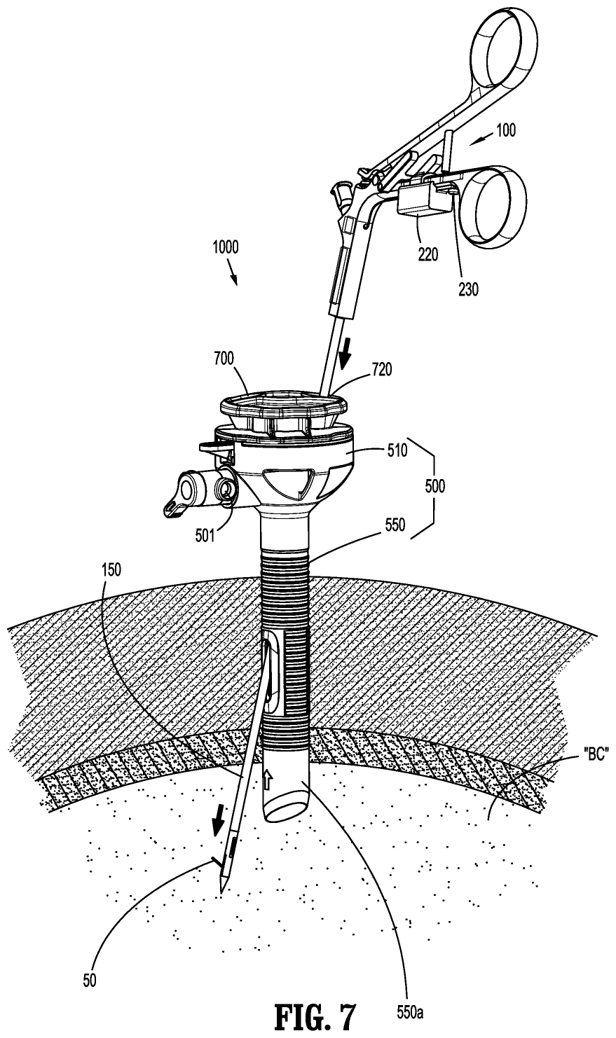

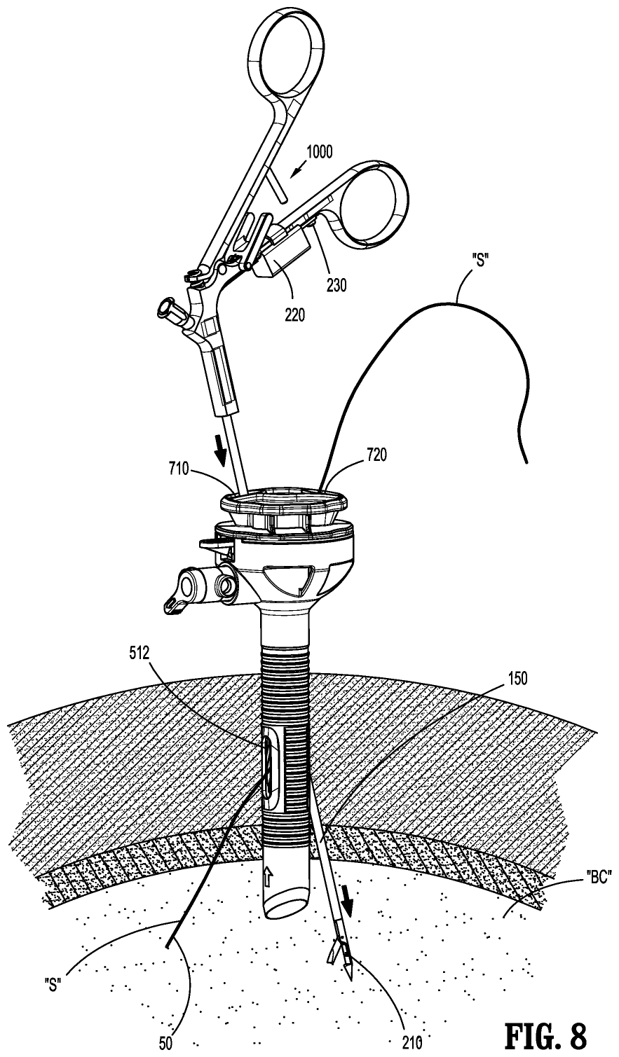

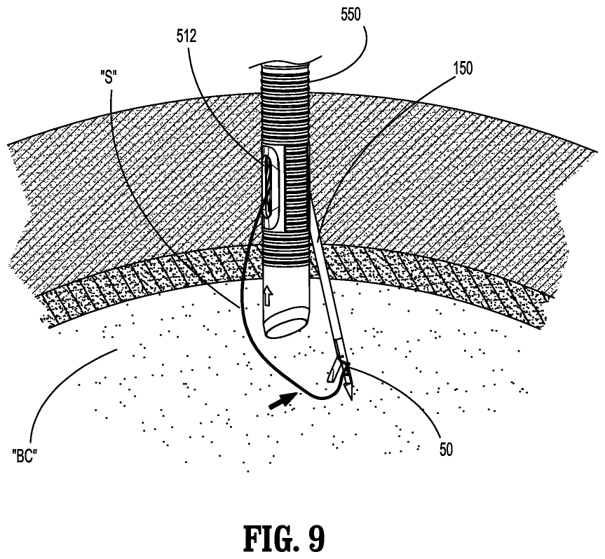

With respect to FIG. 1, a suture passer in accordance with the disclosure is shown generally as a suture passer 100. The suture passer 100 may be part of a fascial closure system 1000 (FIG. 6) utilized to close an incision through which a surgical access device 500 (FIG. 6) is inserted into the body during a surgical procedure. The suture passer 100 includes a handle assembly 120, an elongate member 150, a jaw assembly 180, and a suture retrieval assembly 200 that enables a clinician to retrieve a suture “S” (FIG. 5) within a body cavity “BC” (FIG. 7), thereby eliminating a need for additional surgical instruments such as, e.g., a grasper, to retrieve the suture “S”. The handle assembly 120 is operably coupled to the jaw assembly 180 to transition the jaw assembly 180 between open and closed configurations and activate an electromagnet 210 (FIG. 3) of the suture retrieval assembly 200 disposed in the jaw assembly 180. The suture retrieval assembly 200 enables the clinician to electromagnetically attach a highly magnetic metal portion 50 of the suture “S” to the jaw assembly 180.

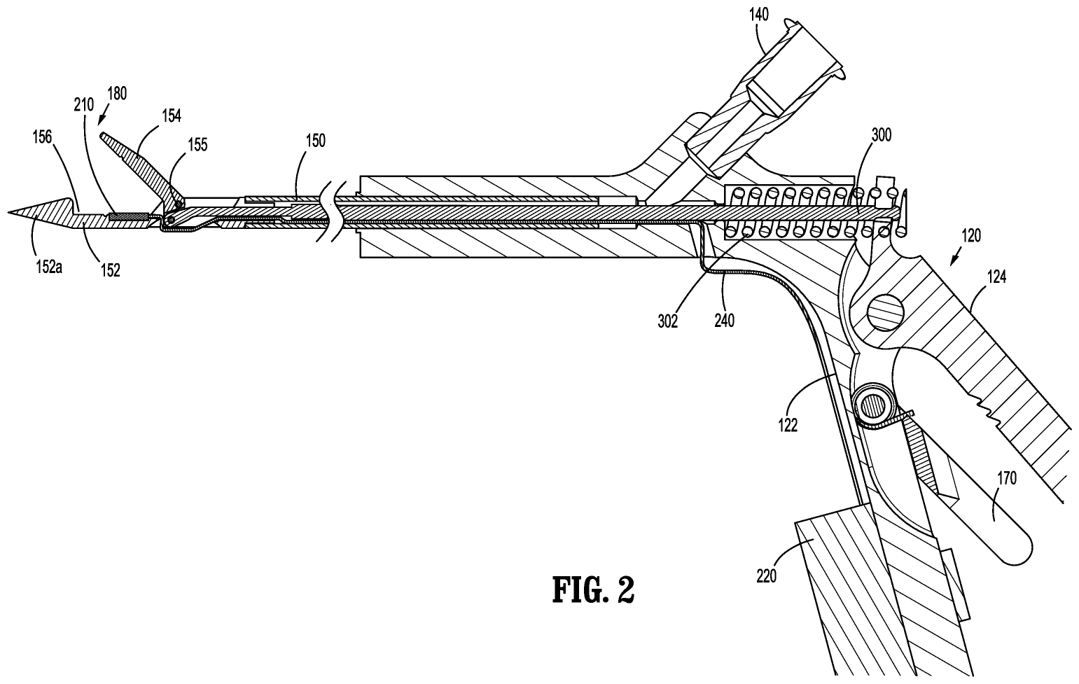

FIGS. 1 and 2 illustrate the handle assembly 120 including a stationary handle 122 and a movable handle 124 pivotably coupled to the stationary handle 122. The movable handle 124 is operably coupled to an actuation rod 300 slidably extending through the elongate member 150. The actuation rod 300 interconnects the jaw assembly 180 and the handle assembly 120. In particular, the movable handle 124 is pivotably supported on the stationary handle 122 such that when the movable handle 124 is displaced relative to the stationary handle 122, the actuation rod 300 is axially displaced. The handle assembly 120 includes a spring 302 to bias the actuation rod 300 to a distal-most position. The handle assembly 120 further includes a fluid port 140 in communication with the jaw assembly 180 to supply a fluid thereto. The elongate member 150 extends distally from the handle assembly 120 and operatively supports the jaw assembly 180 at a distal end portion 150a of the jaw assembly 150. The elongate member 150 defines a lumen dimensioned to receive the actuation rod 300 therethrough. The handle assembly 120 may further include a locking mechanism 170 to secure the jaw assembly 180 in position once the jaw assembly 180 is transitioned to the closed position to hold the suture “S” between the stationary and movable jaws 152, 154.

FIGS. 1-3 further illustrate the jaw assembly 150 including a stationary jaw 152 and a movable jaw 154 pivotably supported on the stationary jaw 152 about a pivot 155. Further, the movable jaw 154 is pivotably coupled to the actuation rod 300 such that axial displacement of the actuation rod 300 transitions the movable jaw 154 towards or away from the stationary jaw 152. The stationary jaw 152 includes a pointed tip 152a having, e.g., a conical profile, to facilitate penetration into tissue. Further, the stationary jaw 152 defines a recess 156 dimensioned to receive the movable jaw 154 therein. The stationary and movable jaws 152, 154 may define respective grooves 152b, 154b to receive, e.g., the highly magnetic metal portion 50, of the suture “S” therein.

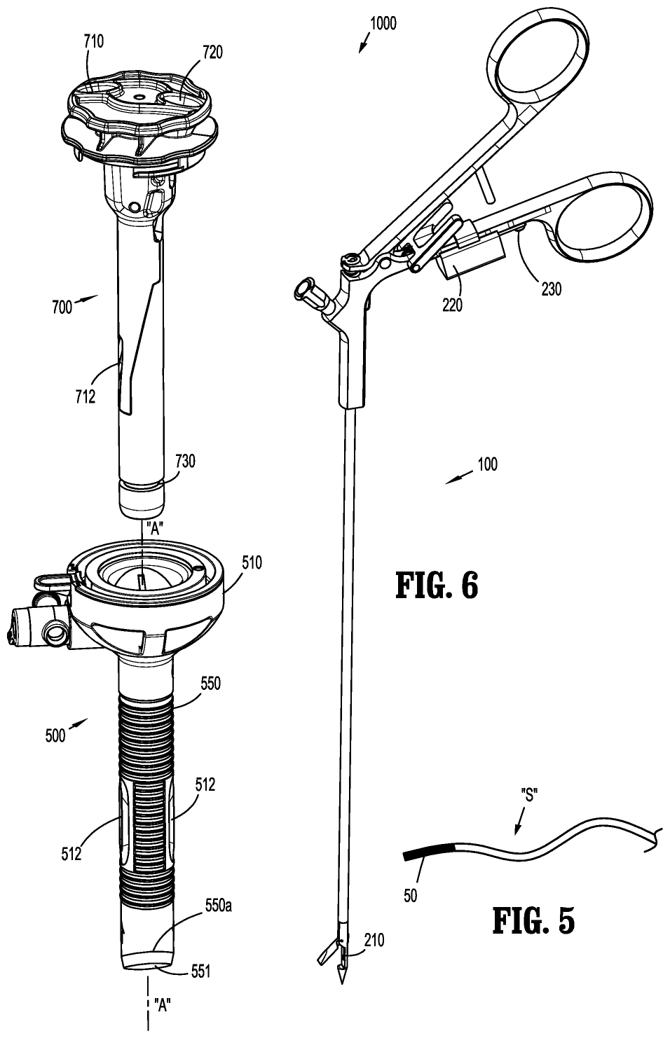

FIGS. 1-4 illustrates the suture passer 100 further including the suture retrieval assembly 200. The suture retrieval assembly 200 enables the jaw assembly 180 to electromagnetically attract the highly magnetic metal portion 50 of the suture “S” (FIG. 5) to facilitate grasping of the suture “S” by the jaw assembly 180. In this manner, a need for additional surgical instruments such as a secondary suture grasper is eliminated, thereby reducing trauma caused by the additional surgical instrument. The suture retrieval assembly 200 includes the electromagnet 210, a power module 220, an activation button 230, and a wire 240 electrically connecting the electromagnet 210, the power module 220, and the activation button 230. The electromagnet 210 includes a planar surface to facilitate grasping of suture “S” thereon. The electromagnet 210 is operatively associated with the jaw assembly 180. In particular, the electromagnet 210 may be disposed in the recess 156 of the stationary jaw 152. The power module 220 may be disposed on the handle assembly 120. In particular, the power module 220 may be secured to the stationary handle 122 and the activation button 230 may be ergonomically positioned along the stationary handle 230. The power module 220 may include a battery and a control unit including a printed circuit board having a processor. The activation button 230 may be coupled to the power module 220 to selectively supply current to the electromagnet 210 to attract the suture “S” thereto. In order for the electromagnet 210 to attract the suture “S”, the suture “S” may include a highly magnetic metal portion 50 (FIG. 5). For example, the highly magnetic metal portion 50 of the suture “S” may be formed of nickel, cobalt, or any combination thereof.

FIG. 6 illustrates the fascial closure system 1000 to close an incision through which a surgical access device is inserted into the body. The fascial closure system 1000 includes the suture passer 100, a surgical access device 500, and a guide 700. The surgical access device 500 includes a housing 510 and a cannula 550 extending distally from housing 510. The cannula 550 defines a lumen 551 therethrough and lateral slots 512 opposing each other. The guide 700 is configured to selectively engage the housing 510. The guide 700 includes a first channel 710 and a second channel 720 extending therethrough, and a seal 730 (e.g., an O-ring) configured to engage an inner wall of cannula 550 for establishing a fluid-tight boundary in the cannula 550 that maintains a pneumoperitoneum in a patient. In addition, the first and second channels 710, 720 are dimensioned to receive the suture passer 100 therethrough. The guide 700 defines lateral openings 712 (only one shown) that diametrically oppose each other. In particular, the first and second channels 710, 720 are in communication with the respective lateral openings 712 such that the first and second channels 710, 720 define an acute angle with respect to a longitudinal axis defined by the guide 700. Further, when the guide 700 is positioned with the surgical access device 500, i.e., at least a portion of the guide 700 is received within the surgical access device 500, the lateral openings 712 of the guide 700 are in registration with the respective lateral slots 512 of the surgical access device 500 such that the suture passer 100 inserted through the first or second channels 710, 720 of the guide 700 extends through the corresponding lateral slot 512 of the surgical access device 500. Further, while housings of a surgical access device may typically include seals therein, the housing 510 described herein is not a typical seal housing, as such a seal housing is generally removed from housing 510 prior to insertion of the guide 700 through the housing 510 and the cannula 550. It is contemplated that the surgical access device 500 may include an expandable member or balloon fixation device (not shown) disposed adjacent a distal end portion 550a of the cannula 550 to improve securement of the access device 500 with a body cavity and/or to provide a sealing relation therewith. In this manner, the suture passer 100 may be utilized in conjunction with the surgical access device 500 and the guide 700 to pass the suture “S” through the cannula 550 and to tissue adjacent an incision.

In use, the distal end portion 550a (FIG. 7) of cannula 550 of the surgical access device 500 is positioned within a patient (e.g., in the abdominal cavity). At this time, the body cavity “BC” (FIG. 7) may be inflated by supplying insufflation fluid to the body cavity “BC” through an inflation port 501 (FIG. 7). A desired surgical procedure is performed utilizing surgical instruments inserted through a lumen of the cannula 550. Following the surgical procedure, the surgical instruments are removed from lumen, and a seal housing, when present, is removed from engagement with the housing 510 of the surgical access device 500. Next, the guide 700 is placed in registration with the surgical access device 500 such that the lateral openings 712 of the guide 700 and the lateral slots 512 of the surgical access device 500 are in registration with each other. Thereafter, a portion such as, e.g., the highly magnetic metal portion 50, of the suture “S” is grasped between the stationary and movable jaws 152, 154 of the suture passer 100. In this manner, the suture “S” and the suture passer 100 are inserted through one of the first or second channels 710, 720 of the guide 700 and are extended through the corresponding lateral slot 512 of the cannula 550. (FIG. 7 illustrates the suture passer 100 inserted through the second channel 720). Under such a configuration, the elongate member 150 of the suture passer 100 defines an acute angle with respect to the longitudinal axis “A-A” (FIG. 6) defined by the cannula 550. Once the highly magnetic metal portion 50 of the suture “S” is placed within the body cavity “BC”, as shown in FIG. 7, the suture “S” is released from the suture passer 100 and the suture passer 100 is retracted and removed from the guide 700 through the second channel 720 of the guide 700. Thereafter, the suture passer 100 is inserted into the other one of the opposing first or second channel 710, 720 of the guide 700 and extends out of the corresponding lateral slot 512 of the cannula 550. (FIG. 8 illustrates the suture passer 100 inserted through the first channel 710). Once the jaw assembly 180 of the suture passer 100 is disposed adjacent the distal end portion 550a of the cannula 550 of the surgical access device 500, the clinician may transition the jaw assembly 180 to the open configuration such that the stationary jaw 152 and the movable jaws 154 are spaced apart, thereby exposing the electromagnet 210 disposed in the recess 156 of the stationary jaw 152. At this time, the clinician may activate the power module 220 to supply current to the electromagnet 210 by pressing the activation button 230 on the stationary handle 122.

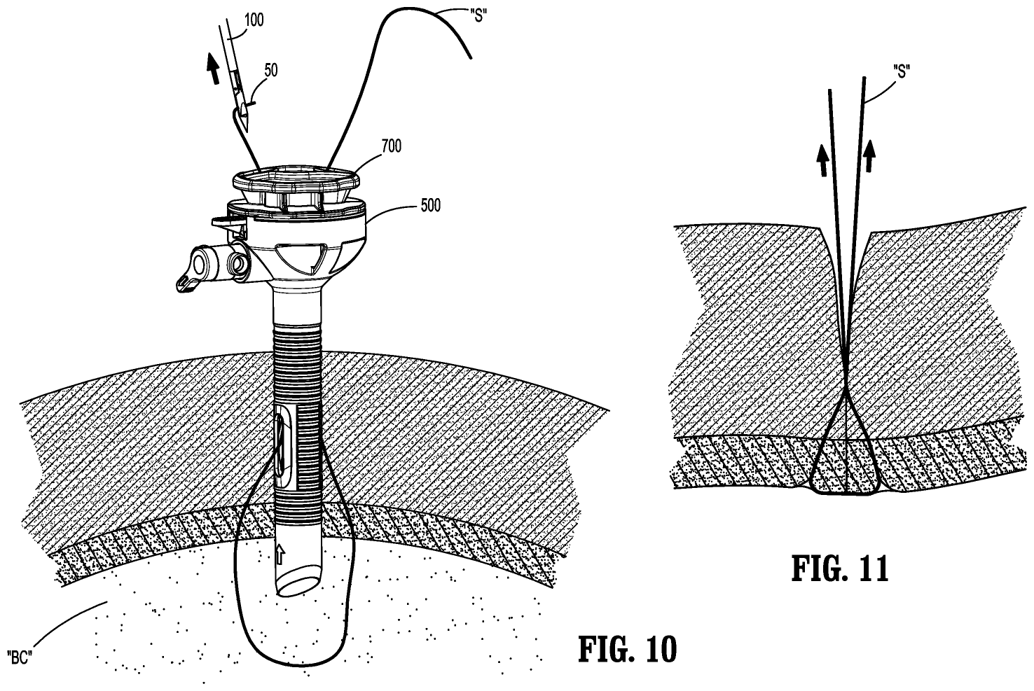

Activation of the electromagnet 210 attracts the highly magnetic metal portion 50 of the suture “S” to the electromagnet 210, as shown in FIG. 9. In this manner, the suture “S” may be retrieved and grasped by the jaw assembly 180 without utilizing a secondary grasper, thereby reducing additional entrance wound or trauma to the patient. The clinician may secure the suture “S” with the jaw assembly 180 by transitioning the jaw assembly 180 to the closed configuration to clamp the suture “S” between the stationary and movable jaws 152, 154. At this time, the highly magnetic metal portion 50 and the suture passer 100 may be retracted and removed from the guide 700 through the first channel 710 of the guide 700, as shown in FIG. 10. With both ends of the suture “S” out of the body cavity “BC”, the clinician may, e.g., tie a knot, to close the incision or the wound, as shown in FIG. 11. The clinician may repeat this process as needed in performing fascial closure.

It will be understood that various modifications may be made to the disclosed suture passer and the fascial closure system. Therefore, the above description should not be construed as limiting, but merely as exemplifications of the disclosure. Those skilled in the art will envision other modifications within the scope and spirit of the disclosure.

1. A surgical kit comprising:
a suture passer including:
a handle assembly;
an elongate shaft extending from the handle assembly and defining a lumen therethrough;
a jaw assembly including first and second jaws transitionable between open and closed configurations; and
a suture retrieval assembly including:
an electromagnet disposed in the jaw assembly;
a power module electrically coupled to the electromagnet; and
an activation switch disposed on the handle assembly and electrically coupled to the power module to selectively activate the electromagnet independently of transitioning the first and second jaws between open and closed configurations;
a suture including a metal portion;
a surgical access device including a housing and a cannula extending distally from the housing, the cannula defining a lumen therethrough and lateral slots in communication with the lumen, the lateral slots diametrically opposing each other; and
a guide detachably positionable within the surgical access device, the guide including first and second channels and defining lateral openings in communication with the respective first and second channels, the lateral openings being in registration with the respective lateral slots of the surgical access device.

2. The surgical kit according to claim 1, wherein the first jaw is stationary relative to the elongate shaft.

3. The surgical kit according to claim 1, wherein the first jaw includes a sharp tip configured to penetrate tissue.

4. The surgical kit according to claim 1, wherein the first jaw defines a recess, the electromagnet disposed in the recess.

5. The surgical kit according to claim 1, wherein the electromagnet includes a planar surface.

6. The surgical kit according to claim 2, wherein the second jaw is pivotably supported on the first jaw.

7. The surgical kit according to claim 1, wherein the first or second channels of the guide defines an acute angle with respect to a longitudinal axis defined by the guide.

8. The surgical kit according to claim 2, wherein the handle assembly includes a fluid port in communication with the jaw assembly.

9. The surgical kit according to claim 1, wherein the power module includes a battery and a printed circuit board including a processor.

10. A suture passer for fascial closure comprising:
a handle assembly including a pair of handles;
an elongate shaft extending from the handle assembly and defining a lumen therethrough;
a jaw assembly including first and second jaws transitionable between open and closed configurations;
an actuation rod interconnecting the pair of handles and the jaw assembly such that actuation of the pair of handles transitions the jaw assembly between the open configuration and the closed configuration;
a suture including a metal portion; and
a suture retrieval assembly including an electromagnet associated with the jaw assembly, a power module in electrical communication with the electromagnet, and an activation switch to selectively activate electromagnet to attract the metal portion of the suture thereto irrespective of a position of the actuation rod.

11. The suture passer according to claim 10, wherein the electromagnet is disposed on one of the first or second jaws.

12. The suture passer according to claim 10, wherein the pair of handles includes a stationary handle and a movable handle pivotably supported on the stationary handle, the power module of the suture retrieval assembly disposed on the stationary handle.

13. The suture passer according to claim 10, wherein the first jaw is fixedly secured to the elongate shaft and the second jaw is pivotably supported on the first jaw.

14. The suture passer according to claim 10, wherein the first jaw has a pointed tip to penetrate tissue.

15. The suture passer according to claim 10, wherein the first jaw defines a recess configured to receive the electromagnet.

16. The suture passer according to claim 10, wherein the electromagnet has a planar surface.

17. The suture passer according to claim 10, wherein the metal portion of the suture is formed of nickel or cobalt.

18. A suture passer comprising:
a handle assembly including first and second handles;
an elongate shaft extending from the handle assembly and defining a lumen therethrough;
a jaw assembly including first and second jaws transitionable between open and closed configurations; and
a suture retrieval assembly including an electromagnet disposed on the first jaw, a battery disposed on the first handle and in electrical communication with the electromagnet, and an activation switch to selectively activate the electromagnet independently of transitioning the first and second jaws between open and closed configurations.

19. The suture passer according to claim 18, wherein the electromagnet includes a planar surface.

20. The suture passer according to claim 18, wherein the activation switch is disposed on the first handle of the handle assembly.
